(19) United States
(12) Patent Application Publication
(10) Pub. No.: US 2005/0019388 A1
(43) Pub. Date: Jan. 27, 2005

(54) Edible capsules

(75) Inventors: Kiyonori Furuta, Kawasaki-shi (JP); Kentarou Maruyama, Kawasaki-shi (JP); Yukiko Takakura, Kawasaki-shi (JP); Hiroyuki Sato, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Ajinomoto Co., Inc., Tokyo (JP)

(21) Appl. No.: 10/866,142

(22) Filed: Jun. 14, 2004

Related U.S. Application Data
(63) Continuation of application No. PCT/JP02/12879, filed on Dec. 9, 2002.

(30) Foreign Application Priority Data
Dec. 11, 2001 (JP) 2001-377163
Mar. 13, 2002 (JP) 2002-068569

(51) Int. Cl. A61K 9/48

(52) U.S. Cl. 424/452

(57) ABSTRACT

The present invention provides an edible capsule for use in the pharmaceuticals, foods, health foods fields, which is composed of non-animal material and can be ingested. The present invention further provides an acid-resistant capsule that comprises non-animal materials.

### CROSS-REFERENCE TO RELATED APPLICATIONS

The present application is a continuation of International Patent Application No. PCT/JP02/12879, filed on Dec. 9, 2002, which claims priority to Japanese Patent Application No. 377163/2001, filed on Dec. 11, 2001, and to Japanese Patent Application No. 068569/2002, filed on Mar. 13 2002. The entire contents of each of these applications are incorporated herein by reference.

### BACKGROUND OF THE INVENTION

1. Field of the Invention

The present invention provides an edible capsule that is utilized in the fields of pharmaceuticals, foods, and health foods. The edible capsule of the present invention may also be used in feeds for animals, as well as in the fields of cosmetics, toiletry, and bath goods. The edible capsule of the present invention is prepared from non-animal materials using polyglutamic acid and/or a salt thereof.

The present invention further provides an acid-resistant capsule that comprises non-animal materials. The acid-resistant capsule of the present invention is prepared from polyglutamic acid and/or a salt thereof where weight-average molecular weight of the polyglutamic acid and/or a salt thereof is more than 300,000.

2. Discussion of the Background

Edible capsules used in the pharmaceuticals and foods fields include: (a) hard capsules that are suitable for enclosing powdery or granular pharmaceuticals, foods, etc. and (b) soft capsules that are suitable for enclosing water-insoluble liquids, such as oil liquid, pasty oil liquid and oil liquid in which powder is suspended. In addition, several microcapsules that have a diameter of several μm to several hundred μm are known.

As a result of increasing societal health consciousness over the recent years, health foods such as a dietary supplements, a functional food, have been receiving public attention. Various forms of materials have been used for health foods including oily substances and powders. Capsules have been used for protection and stabilization of the components contained in these health foods. Further, the capsules may be used to mask taste and smell thereof.

Hard capsules are mostly used for filling of powder, but filling of liquid substance therein is difficult. In the case of soft capsules, there are advantages that both liquid substance and powder may be filled and that various shapes can be selected by changing the mold tool. In view of the above, soft capsules are widely used in addition to hard capsules in the field of health foods. Moreover, microcapsules are widely used for the purpose of protection of unstable substances such as perfume, control of release of pharmaceuticals such asDDS, enclosure of substances having unusual taste and unusual flavor in food, etc.

In contrast, in the conventional edible capsules, gelatin derived from animals such as cattle, swine, bird and fish have been used for a shell substrate thereof while there has been an increasing demand from the market for non-animal materials. Therefore, the present inventors have conducted investigations for the use of non-animal materials such as agar material. Arch material and other various saccharides as materials for capsule shell but several problems such as that method for their manufacture is complicated, existing equipments are unable to be used, shell is hard and unable to be spread and shape of the capsule is limited have been pointed out and, therefore, they do not satisfy the characteristic required for capsules.

In regard to acid-resistant capsules, a method is known where the surface of a capsule made of gelatin derived from animals is coated with a substance that is insoluble in an acidic solution and a method where gelatin is mixed with a substance that is insoluble in an acidic solution. However, since these methods use gelatin, they are not acid-resistant capsules comprising non-animal material.

In contrast, in regard to a cross-linked product using a polyglutamic acid, JP-A-10-251402 discloses a cross-linking method by means of radical polymerization by irradiation of radioactive ray and JP-A-11-343339 discloses a cross-linking method using a polyepoxy compound. However, these methods suffer in that there are problems such as restriction in terms of equipments and legal restriction for utilization in the field of foods whereby there is a difficulty. In JP-A-2001-181387, a water-absorbing polymer where polyglutamic acid is cross-linked with metal is disclosed although there is no disclosure at all for its specific use, particularly as edible capsules.

Accordingly, the present invention serves to solve the problems listed above.

### SUMMARY OF THE INVENTION

It is an object of the present invention to provide an edible capsule to be used in the pharmaceuticals, foods, health foods fields. In this object of the invention, the edible capsule is composed of non-animal materials that can be ingested.

In another object of the present invention is an acid-resistant capsule that comprises non-animal materials.

The present inventors have found that the above-mentioned problems can be solved when polyglutamic acid and/or a salt thereof are/is used in edible capsules. Thus, the present invention relates to an edible capsule containing polyglutamic acid and/or a salt thereof. The present invention also relates to an acid-resistant edible capsule that contains a weight-average molecular weight of the polyglutamic acid and/or a salt thereof is more than 300,000.

The above objects highlight certain aspects of the invention. Additional objects, aspects and embodiments of the invention are found in the following detailed description of the invention.

### BRIEF DESCRIPTION OF THE FIGURES

A more complete appreciation of the invention and many of the attendant advantages thereof will be readily obtained as the same becomes better understood by reference to the following Figures in conjunction with the detailed description below.

FIG. 1 is a photographic image of the microcapsule of Example 5 under a confocal laser-scanning microscope.

FIG. 2 shows the result of measurements of particle size distribution of the microcapsule of Example 5.

FIG. 3 is a photographic image of the microcapsule of Example 6 under a confocal laser-scanning microscope.

FIG. 4 shows the result of measurements of particle size distribution of the microcapsule of Example 6.

### DETAILED DESCRIPTION OF THE INVENTION

Unless specifically defined, all technical and scientific terms used herein have the same meaning as commonly understood by a skilled artisan in the fields of chemistry, pharmaceuticals, foods, health foods, cosmetics, and personal hygiene products.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

In regard to polyglutamic acid and/or a salt thereof used in the present invention, anything may be used without particular limitation so far as it is a compound where glutamic acid is polymerized although poly-γ-glutamic acid and/or a salt thereof are/is preferred.

Polyglutamic acid can be manufactured by chemical synthesis and enzymatic synthesis and also by means of incubation using cells of genus Bacillus such as Bacillus natto. When B. natto or the like is used, polyglutamic acid contained in viscous substance of natto is preferably extracted and used or polyglutamic acid, which is extracellularly secreted from the cells, is used.

Generally, polyglutamic acid in the viscous substance of natto or polyglutamic acid secreted by genus Bacillus under the conventional incubation condition is a high-molecular substance. Further, the polyglutamic acid itself is highly viscous and, therefore, it can be treated to reduce its molecular weight by an acid or an enzyme depending upon the use.

For the purpose of releasing the content of the edible capsule after being dissolved in stomach, it is preferred to prepare polyglutamic acid having a molecular weight of 10,000 to 300,000 by making into low molecules using an acid or an enzyme.

For the purpose of releasing the content of the edible capsule that is not resistant to an acid in small intestine or the like, it is necessary to give an acid-resistant property to the same. In this regard, the edible capsule may be hardly soluble in the stomach, which has a highly acidic pH, while easily soluble in digestive tract such as small intestine having a neutral pH. For such a use, it is preferred that the molecular weight of polyglutamic acid is more than 300,000.

Incidentally, the molecular weight used in the present invention is a weight-average molecular weight measured by a gel filtration—light scattering method (GPC-MALLS method; Dawn DPS manufactured by Wyatt Technology).

Polyglutamic acid used in the present invention may be in a free or a salt form. Polyglutamate can be manufactured by the reaction of polyglutamic acid with a basic compound. At that time, there is no particular limitation for the basic compound but an alkaline metal or alkaline earth metal hydroxide such as sodium hydroxide, potassium hydroxide or magnesium hydroxide or an organic basic compound such as ammonia or amine may be used. In regard to the ratio in the reaction of the basic compound with polyglutamic acid, the reaction of the basic compound within a range of 0.1 to 1.0 equivalents may be performed to one equivalent of carboxylic acid in polyglutamic acid.

In regard to the metal used for the formation of a cross-linked product of polyglutamic acid, a divalent or higher metal, which can react with plural carboxyl groups in polyglutamic acid, is used. Preferably, calcium, iron, aluminum, chromium, etc. may be used.

It is also possible to use a metal compound containing such a metal. Although there is no particular limitation for the metal compound, it is preferred to use a calcium compound, an aluminum compound and/or a double salt thereof. More specifically, calcium chloride, calcium hydroxide, calcium carbonate, aluminum potassium sulfate, aluminum potassium sulfate dodecahydrate (potassium alum), aluminum ammonium sulfate, and aluminum ammonium sulfate dodecahydrate (ammonium alum) may be used.

In regard to polyglutamic acid or the like and the above metal compound, mixing of the same may be performed in a rate of 100 parts by weight of polyglutamic acid and 1 mmol to 100 mmol of the metal compound or, preferably, 5 mmol to 75 mmol. Such a mixing rate varies depending upon the molecular weight of polyglutamic acid and the species of the metal whereby it may be appropriately decided.

Addition of polyglutamic acid or a cross-linked product of polyglutamic acid, a plasticizer and water, may obtain the polyglutamic acid shell that constitutes the edible capsules of the present invention. Examples of the plasticizer are sorbitol, mannitol, glycerol and 1,3-butylene glycol. Although addition of the plasticizer is not essential, it is added usually within a range of from 5 to 60 parts or, preferably, from 10 to 50 parts by weight to 100 parts by weight of polyglutamic acid. Additional optional components that may be added to the polyglutamic acid shell if necessary include: a disintegration promoter, a stabilizer, a coloring agent, and perfume. In addition, shell materials for capsules that have been known and used already may be also used together therewith.

The edible capsule of the present invention may be adapted to any of various capsules such as hard capsules, soft capsules, seamless soft capsules, and microcapsules.

In regard to a method for manufacturing hard capsules, a dipping method where a mold tool (mold pin) is dipped in a solution containing shell substrate followed by drying, etc. may be used.

In regard to a method for manufacturing soft capsules, a rotary die method that is a kind of a stamping method may be used. The rotary die method is a method where two sheets are used and molding of the capsule, filling of the content and heat sealing are performed at the same time.

In regard to a method for manufacturing seamless soft capsules, a hardening-in-the-liquid method that is a kind of a dripping method may be used. This is a method where an content liquid for capsule and a solution containing shell substrate flow out at a predetermined rate from inner and outer nozzles, respectively, in the double or more nozzles, the liquid flow in two layers as such is cut with a predetermined interval to give liquid droplets and then a shell layer for the outside is gelled to make into a capsule.

In regard to a method for manufacturing microcapsules, a coacervation method (a phase separation method), an emulsion method (a stirring emulsifying method, an ultrasonic emulsifying method), a spray-drying method, etc. may be used.

In regard to shape, size, etc. of the edible capsule, there is no particular limitation and, as to the shape, it is possible to manufacture in a round shape, an oval shape, an oblong shape, a tube shape, a teardrop shape, etc. As to the size, it is possible to manufacture the capsule in a size from about several μm to about several cm.

As mentioned above, the manufacturing method may be performed using a conventionally known manufacturing methods depending upon the intention for use, the necessary shape and size of the edible capsule.

Incidentally, according to Japanese Patent Laid- Open No. 03/030,648, Japanese Patent Laid-Open No. 05/316,999, Japanese Patent No. 3,232,718, U.S. Pat. No. 5,447,732 and European Patent No. 605,757, it has been known that poly-γ-glutamic acid contained in a viscous substance of natto has an effect of promoting the absorption of minerals such as calcium and iron into the body. Accordingly, the edible capsule of the present invention containing polyglutamic acid and/or a salt thereof is expected to have a function of promoting the absorption of mineral components filled therein as a content or of mineral components ingested as foods in addition to the function which has been already available in the conventional capsules.

The above written description of the invention provides a manner and process of making and using it such that any person skilled in this art is enabled to make and use the same, this enablement being provided in particular for the subject matter of the appended claims, which make up a part of the original description.

As used herein, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

### Manufacturing Example 1

### Manufacture of Poly-γ-Glutamic Acid (Molecular Weight: 30,000)

Bacillus natto (Accession Number at the National Institute of Bioscience and Human Technology, Agency of Industrial Science and Technology: FERM P-10607) belonging to Bacillus subtilis was subjected to a seed culture at 32° C. for 24 hours in a culture solution (pH 6.0) containing 0.3% of malt extract, 0.3% of yeast extract, 0.5% of polypeptone and 1.0% of glucose using a three-liter mini-jar. Subsequently, 0.5% of the seed-cultured solution was inoculated to a culture solution (pH 6.4) containing 7.5% of glucose, 1.5% of ammonium sulfate, 0.15% of magnesium sulfate, 5.0% of monosodium glutamate and 1.0% of sodium chloride using a 500-liter jar and incubated at 37° C. for 48 hours at 250 rpm and aeration of 0.5 to 1 vvm. After the incubation, the culture solution was subjected to a GPC method to measure the amount of poly-γ-glutamic acid, which was determined to be 3.0 g/dl. The resulting culture solution (50 liters) was adjusted to pH 2 using a concentrated hydrochloric acid, filtered through a precise filtration membrane (PSP-313 manufactured by Asahi Chemical Industry; pore size: 0.1 μm; membrane area: 6.0 m²) and heated at 50° C. for 6 hours to reduce the molecular weight.

After cooling, the filtered culture solution was neutralized with sodium hydroxide and desalted using an ultrafilter membrane (SIP-3013 manufactured by Asahi Chemical Industry; fractionating molecular weight: 6,000; membrane area: 4.7 m²) with addition of water in the same amount as the permeated solution to the circulating solution. When the amount of the water added reached 50 liters, desalting was considered to be complete and the solution was concentrated until the amount of e circulating solution reached 8 liters. This concentrated solution was further concentrated in vacuo to give an aqueous solution of poly-γ-glutamic acid. Its concentration by a GPC method was 16% and weight-average molecular weight by a GPC-MALLS method was 30,000. The resulting aqueous solution of poly-γ-glutamic acid was dried using a vacuum drum drier (drum heating temperature: 110° C.; degree of vacuation: 10 Torr; drying time: 30 seconds) to give pole-γ-glutamic acid.

### Manufacturing Example 2

### Manufacture of Poly-γ-Glutamic Acid (Molecular Weights: 260, 000, 490,000 and 800,000)

After the culture solution was prepared according to the method described in Manufacturing Example 1, the pH was adjusted and the solution was precisely filtered. The same operation as in manufacturing Example 1 was performed except that the treatment for making the molecular weight low was at 50° C. for 0, 10 or 30 minute(s) to give poly-γ-glutamic acid. Weight-average molecular weights by a GPC-MALLS method were 800,000, 490,000 and 260,000, respectively.

Characteristic values shown in Examples and Comparative Examples were measured and judged according to the following methods.

<Appearance>

It was judged by naked eye.

<Appropriateness for Molding>

It was judged by naked eye whether molding of capsules was possible.

<Viscosity of the Solution Containing Shell Substrate>

It was measured at 25° C. using a viscometer of type B (manufactured by K. K. Tokyo Keiki).

<Water Content of the Shell>

It was measured under the condition of 105° C. for 2 hours using an infrared device for measuring the water content (Kett FD-600).

<Solubility Test>

A capsule was poured into water that was warmed at 40±1° C. and allowed to stand, the dissolving state of the capsule was observed and time until it was completely dissolved was measured.

<Disintegration Test>

In accordance with the disintegration test method mentioned in the Japanese Pharmacopoeia, a disintegration test was performed according to the following methods using the first solution (artificial gastric juice) and the second solution (artificial intestinal juice).

1. Test solution

First solution: Sodium chloride (2 g), 7 ml of hydrochloric acid and water were mixed and dissolved to make one liter (pH 1.2).

Second solution: A 0.2 mol/liter sodium hydroxide reagent ( 118 ml) and water were added to 250 ml of a 0.2 mol/liter potassium dihydrogen phosphate reagent to make one liter (pH 6.8).

2. Test solution temperature and device: Temperature of the test solution: 37±1° C.; Disintegration test device: NT-4HSF manufactured by Toyama Sangyo

3. Criteria for the judgment

Opening time: time until the content came out from the connected part Disintegration time: time until all of the shell was dissolved ⊙=completely dissolved within five minutes; ∘=completely dissolved within ten minutes; ×=10 minutes or more were need for complete dissolving

### Example 1

A solution containing a cross-linked substance of poly-γ-glutamic acid as shell substrate was prepared according to the following composition. Poly-γ-glutamic acid, deionized water and glycerol were added using a one-liter beaker followed by stirring. Then a 6N hydrochloric acid was added with stirring to prepare a poly-γ-glutamic acid solution. Further, 0.2M aluminum potassium sulfate dodecahydrate was added thereto followed by stirring and then a 10N sodium hydroxide was added thereto followed by stirring. When sodium hydroxide was added followed by stirring, viscosity of the solution simultaneously increased. When no more change in the viscosity was noted, stirring was finished to give a solution containing a cross-linked substance of poly-γ-glutamic acid as shell substrate.

| | |
|---|---|
| Poly-γ-glutamic acid of Manufacturing Example 1 | 160 parts |
| Deionized water | 200 parts |
| Glycerol (special reagent grade; | 40 parts |
| manufactured by Wako Pure Chemicals) | |
| 6 N Hydrochloric acid (special reagent grade; | 48 parts |
| manufactured by Wako Pure Chemicals | |
| 0.2 M Aluminum potassium sulfate dodecahydrate | 125 parts |
| 10 N Sodium hydroxide | 21 parts |

Viscosity of the above solution containing shell substrate was 12,400 cps. The solution containing shell substrate was defoamed in vacuo, coated on a glass plate using an applicator of 2 mm thickness and air-dried for 24 hours in a chamber kept at constant temperature and humidity of 25° C. and 45% RH. Thickness and water content of the resulting sheet were 0.7 mm and 23%, respectively.

The sheet was made into an edible capsule according to a conventional method using a rotary capsule molding machine (manufactured by Kamata Co., Ltd.). In regard to a metal mold, No. 5 of an oval type was used while, in regard to a filler, soybean oil was used. The resulting sheet containing a cross-linked poly-γ-glutamic acid substance could be made into good capsules. It was also made clear that the edible capsules using a cross-linked poly-γ-glutamic acid substance had the same appearance and molding adaptability as those of the conventional edible capsules using gelatin.

Subsequently, the resulting edible capsules were allowed to stand in water of 40° C. and a dissolving test was performed whereupon all shells were dissolved within 16 minutes and the contents were discharged.

### Example 2

A solution containing a cross-linked substance of poly-γ-glutamic acid as shell substrate was prepared according to the following composition. Poly-γ-glutamic acid, deionized water and glycerol were added using a one-liter beaker followed by stirring. Then a 6N hydrochloric acid was added with stirring to prepare a poly-γ-glutamic acid solution. Further, 0.2M aluminum potassium sulfate dodecahydrate was added thereto followed by stirring and then a 10N sodium hydroxide was added thereto followed by stirring. When sodium hydroxide was added followed by stirring, viscosity of the solution simultaneously increased. When no increase in the viscosity was noted, stirring was finished to give a solution containing a cross-linked substance of poly-γ-glutamic acid as shell substrate.

| | |
|---|---|
| Poly-γ-glutamic acid of Manufacturing Example 1 | 170 parts |
| Deionized water | 102 parts |
| Glycerol (special reagent grade; | 43 parts |
| manufactured by Wako Pure Chemicals) | |
| 6 N Hydrochloric acid (special reagent grade; | 85 parts |
| manufactured by Wako Pure Chemicals) | |
| 0.2 M Aluminum potassium sulfate dodecahydrate | 170 parts |
| 10 N Sodium hydroxide | 42.5 parts |

The solution containing shell substrate was warmed at 60° C. and defoamed in vacuo, coated on a flat plate to which a mold-releasing tape was adhered using an applicator of 3 mm thickness and air-dried for 24 hours in a chamber kept at constant temperature and humidity of 22° C. and 25% RH. Thickness and water content of the resulting sheet were 0.7 mm and 23%, respectively. The same operation as in Example 1 was performed except that a medium-chain fatty acid triglyceride was used as a filler whereupon edible capsules were prepared.

The resulting sheet could be made into good capsules. It was also made clear that the edible capsules using a cross-linked poly-γ-glutamic acid substance had the same appearance and molding adaptability as those of the conventional edible capsules using gelatin.

Subsequently, the resulting edible capsules were allowed to stand in water of 40° C. and a dissolving test was performed whereupon all shells were dissolved within 16 minutes and the contents were discharged.

Results of the disintegration test for the edible capsules of Example 2 (mean values of six samples) are shown in Table 1.

TABLE 1

| | | | Comparative |
|---|---|---|---|
| Test Solutions | | Example 2 | Example |
| Artificial | Opening Time | 3 minutes and 37 | 3 minutes and |
| Gastric Juice | | seconds | 19 seconds |
| | Disintegration Time | ◯ | ◯ |
| Artificial | Opening Time | 2 minutes and 37 | 4 minutes and |
| Intestinal | | seconds | 22 seconds |
| Juice | Disintegration Time | ⊚ | X |

### Comparative Example

A solution containing shell substrate was prepared according to the following composition. Deionized water was added to gelatin to swell at room temperature for 1 hour, then glycerol was added thereto and the mixture was heated at 60° C. on a water bath followed by stirring whereupon a gelatin solution was prepared.

| | |
|---|---|
| Gelatin (derived from animal; jelly strength: 150 blooms) | 100 parts |
| Glycerol | 35 parts |
| Deionized water | 100 parts |

The solution containing shell substrate prepared according to the above composition was defoamed in vacuo at 60° C. Thickness of the resulting sheet was 0.85 mm. The same operation as in Examples 1 was performed to give edible capsules constituted from materials derived from animal.

The resulting edible capsules were subjected to a dissolving test by being allowed to stand in water of 40° C. The result was that, even after 30 minutes, the shell and the content remained. Results of the disintegration test of the edible capsules of this Comparative Example (mean values of six samples) are shown in Table 1.

### Example 3

An example for the manufacture of seamless soft capsules by a hardening-in-liquid method will be shown as hereunder.

To 100 parts by weight of poly-γ-glutamic -acid of Manufacturing Example 1 were added 80 parts of deionized water and the pH was adjusted to 4.9 with a 10N aqueous solution of sodium hydroxide to prepare a solution of poly-γ-glutamic acid. To 100 parts of the resulting solution of PGA were added 100 ml of a 2M aqueous solution of calcium chloride to prepare a solution containing poly-γ-glutamic acid as shell substrate. Soybean oil was used as a content liquid while ethanol was used as a hardening solution and a solution containing poly-γ-glutamic acid as shell substrate and the content liquid were dropped into a hardening solution from a double nozzle (diameter of the outer nozzle was 3 mm while that of the inner nozzle was 2 mm). The dropping rate at that time was 4.7 ml/minute for the solution containing poly-γ-glutamic acid as shell substrate and was 3.9 ml/minute for the soybean oil. Spherical capsules were formed. The capsules were taken out from the hardening solution and dried for 12 hours at room temperature to give seamless soft capsules. The resulting seamless soft capsules were allowed to stand in water of 40° C. to carry out a dissolving test. The result was that all shells were dissolved within 3.5 minutes and the content was discharged.

### Example 4

An example for the manufacture of hard capsules using a dipping method will be shown as hereunder.

To 100 parts of poly-γ-glutamic acid (molecular weight: 800,000) obtained in Manufacturing Example 2 were added 700 parts of deionized water and swelling was conducted at room temperature for 30 minutes followed by stirring to give a solution containing poly-γ-glutamic acid as shell substrate. The solution containing shell substrate was allowed to stand at room temperature for 2 hours to defoam. A dipping pin made of stainless steel where the end was rounded was dipped thereinto vertically, removed and dried with hot air of 80° C. by rotating for making the thickness of the solution containing shell substrate uniform until fluidity was no longer noted. After that, it was dried for 12 hours at 25° C. and 45% relatively humidity. The capsule shell obtained after drying was taken out from the dipping pin and cut into a desired length to give an empty capsule. Thickness of the capsule shell was 0.15 mm. The resulting empty capsule was soft and deformable and did not generate crack or the like.

<Acid-resistant test>will be shown as hereunder.

A. To 100 parts of poly-γ-glutamic acid (molecular weight: 260,000) prepared in Manufacturing Example 2 were added 600 parts of deionized water followed by stirring to give a solution containing poly-γ-glutamic acid as shell substrate (A).

B. To 100 parts of poly-γ-glutamic acid (molecular weight: 490,000) prepared in manufacturing Example 2 were added.

600 parts of deionized water and then swelling was conducted at room temperature for 30 minutes followed by stirring to give a solution containing poly-γ-glutamic acid as shell substrate (B).

C. To 100 parts of poly-γ-glutamic acid (molecular weight: 800,000) prepared in Example 4 were added 700 parts of deionized water and then swelling was conducted at room temperature for 30 minutes followed by stirring to give a solution containing poly-γ-glutamic acid as shell substrate(C).

Each of the above solutions (A), (B) and (C) was coated on a glass plate so as to make the membrane thickness after drying 0.15 mm and dried for 12 hours at 25° C. and 45% relative humidity. The sheet was peeled off from the glass plate and cut into a size of 2 mm square to give test pieces.

The test piece (50 mg) was placed in each 50 ml of the first solution (artificial gastric juice) and the second solution (artificial intestinal juice) stipulated by the Japanese Pharmacopoeia and allowed to stand at 37° C. and the time until it was dissolved was measured. The result is shown in Table 2.

TABLE 2

| Test | A (M. W. = | B (M. W. = | C (M. W. = |
|---|---|---|---|
| Solutions | 260,000) | 490,000) | 800,000) |
| Artificial | X | ◯ | ◯ |
| Gastric | | | |
| Solution | | | |
| Artificial | X | X | X |
| Intestinal | | | |
| Solution | | | |

Criteria for judging the acid-resistant solubility:
X = dissolved within 10 minutes
◯ = insoluble during 30 minutes or longer

### Example 5

Microcapsules containing a cross-linked poly-γ-glutamic acid substance were prepared according to the following composition. First 1 deionized water was added to poly-γ-glutamic acid (molecular weight: 26,000), which was separately manufactured by a method of Manufacturing Example 1 so as to make its concentration 10% (w/w) followed by stirring. Subsequently, 27 ml of 10% (w/w) poly-γ-glutamic acid solution and 3 ml of soybean oil were added to a 50-ml stainless steel tube and subjected to an emulsifying dispersion for 1 minute at output of 160 watts using an ultrasonic treating machine (Branson Sonifier 250). After 1 minute, 2 ml of a 0.2M aluminum potassium sulfate dodecahydrate solution were dropped thereinto without stopping the ultrasonic treatment and then an ultrasonic wave treatment was performed for 2 minutes to give a milky white dispersion.

| | |
|---|---|
| 10% (w/w) Poly-γ-glutamic acid solution | 27 parts |
| Soybean oil | 3 parts |
| 0.2 M Aluminum potassium sulfate dodecahydrate | 2 parts |

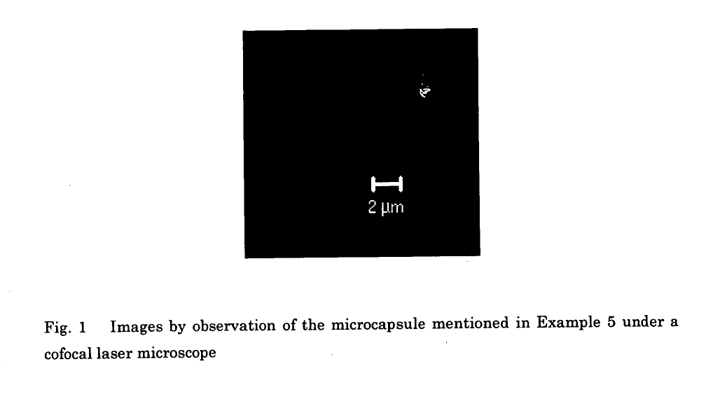

When the above dispersion where the soybean oil was subjected to a fluorescent dyeing with Nile Red while the poly-γ-glutamic acid wa ssubjected to a fluorescent dyeing with Rhodamine was observed under a confocal microscope whereupon the presence of microcapsules enclosing soybean oil therein was confirmed (FIG. 1).

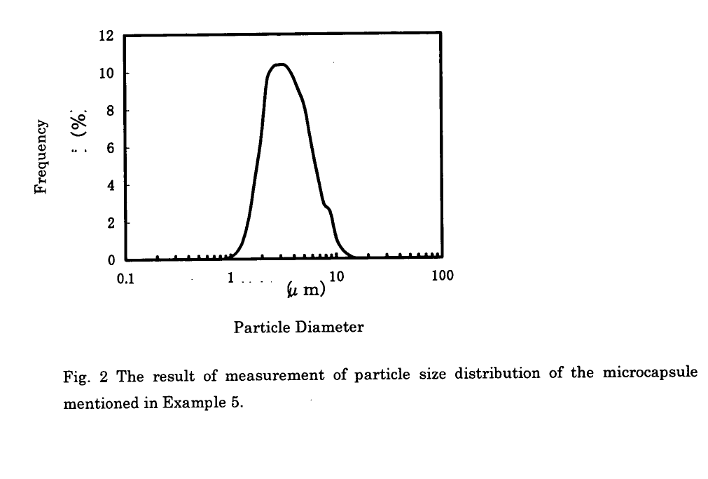

When particle size distribution of the capsule dispersion was measured using a particle size distribution meter of a laser diffraction type (LA 920 of Horiba), a median diameter was so small as 3.2 μm and was a single peak (FIG. 2).

### Example 6

Microcapsules containing a cross-linked poly-γ-glutamic acid substance were prepared according to the following composition. Firstly, deionized water was added to poly-γ-glutamic acid (molecular weight: 26,000) separately manufactured by a method of Manufacturing Example 1 so as to make its concentration 10% (w/w) followed by stirring. After that, 27 ml of 10% (w/w) poly-γ-glutamic acid solution and 3 ml of soybean oil were added to a 50-ml stainless steel tube and subjected to an emulsifying dispersion for 1 minute at 15,000 rpm using a homogenizer (Kinematica PT 3000). After 1 minute, 2 ml of a 0.2M aluminum potassium sulfate dodecahydrate solution were dropped thereinto without stopping the ultrasonic treatment and then an ultrasonic wave treatment was performed for 2 minutes to give a milky white dispersion.

| | |
|---|---|
| 10% (w/w) Poly-γ-glutamic acid solution | 27 parts |
| Soybean oil | 3 parts |
| 0.2 M Aluminum potassium sulfate dodecahydrate | 2 parts |

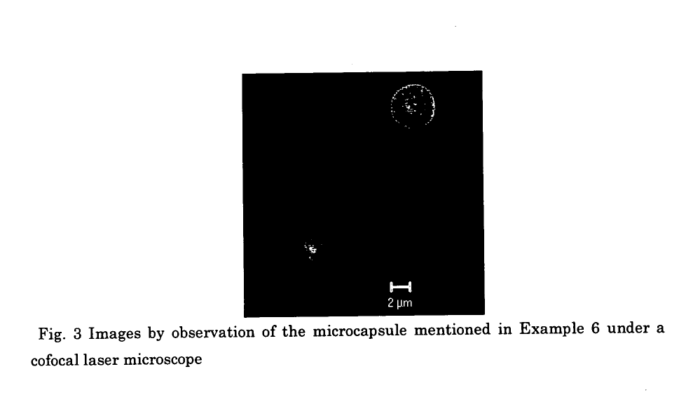

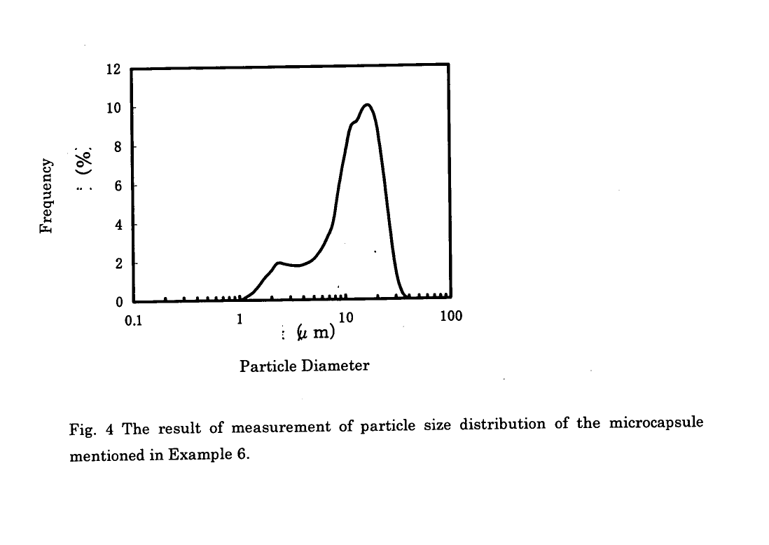

When the above dispersion where the soybean oil was subjected to a fluorescent dyeing with Nile Red while the poly-γ-glutamicacid was subjected to a fluorescent dyeing with Rhodamine was observed under a confocal laser-scanning microscope whereupon the presence of microcapsules enclosing soybean therein was confirmed (FIG. 3). When particle size distribution of the capsule dispersion was measured using a particle size distribution meter of a laser diffraction type (LA 920 of Horiba), a median diameter was 11.7 μm (FIG. 4).
Industrial Applicability

When polyglutamicacid and/or a salt thereof are/is used, it is now possible to give edible capsules being constituted from non-animal materials. The resulting edible capsules have the same appropriateness of molding as conventional capsules and have excellent disintegrating property and solubility and an acid resistance can also be given thereto when molecular weight is duly selected.

Numerous modifications and variations on the present invention are possible in light of the above teachings. It is, therefore, to be understood that within the scope of the accompanying claims, the invention may be practiced otherwise than as specifically described herein.

1. An edible capsule comprising polyglutamic acid, salt of polyglutamic acid, or a mixture of polyglutamic acid and a salt of polyglutamic acid.

2. The edible capsule according to claim 1, wherein said polyglutamic acid, salt of polyglutamic acid, or mixture of polyglutamic acid and a salt of polyglutamic acid is contained in the shell of said edible capsule.

3. The edible capsule according to claim 1, wherein said edible capsule comprises polyglutamic acid.

4. The edible capsule according to claim 1, wherein said edible capsule comprises a salt of polyglutamic acid.

5. The edible capsule according to claim 1, wherein said edible capsule comprises a mixture of polyglutamic acid and a salt of polyglutamic acid.

6. The edible capsule according to claim 1, wherein said polyglutamic acid, salt of polyglutamic acid, or mixture of polyglutamic acid and a salt of polyglutamic acid is cross-linked with a metal compound.

7. The edible capsule according to claim 6, wherein the metal compound is a metal salt or double salt wherein said metal is selected from the group consisting of calcium, iron, aluminum, and chromium.

8. The edible capsule according to claim 6, wherein the metal compound is a metal salt or double salt selected from the group consisting of calcium chloride, calcium hydroxide, calcium carbonate, aluminum potassium sulfate, aluminum potassium sulfate dodecahydrate, aluminum ammonium sulfate, and aluminum ammonium sulfate dodecahydrate.

9. The edible capsule according to claim 6, comprising 1 mmol to 100 mmol of said metal compound per 100 parts by weight of the polyglutamic acid.

10. The edible capsule according to claim 6, comprising 5 mmol to 75 mmol of said metal compound per 100 parts by weight of the polyglutamic acid.

11. The edible capsule according to claim 6, further comprising one or more additional ingredients selected from the group consisting of a plasticizer, a disintegration promoter, a stabilizer, a coloring agent, perfume, and water.

12. The edible capsule according to claim 1, further comprising one or more additional ingredients selected from the group consisting of a plasticizer, a disintegration promoter, a stabilizer, a coloring agent, perfume, and water.

13. The edible capsule according to claim 1, wherein the polyglutamic acid is poly-γ-glutamic acid.

14. The edible capsule according to claim 1, wherein the weight-average molecular weight of the polyglutamic acid ranges from 10,000 to 300,000.

15. The edible capsule according to claim 1, wherein the weight-average molecular weight of the polyglutamic acid is more than 300,000.

16. The edible capsule according to claim 1, wherein said capsule is selected from the group consisting of a hard capsule, a soft capsule, a seamless soft capsule, and a microcapsule.

17. A method of promoting the absorption of mineral components comprising
filling an edible capsule according to claim 1 with a mineral component to obtain a filled edible capsule;
adding the filled edible capsule to a food; and
ingesting the food containing the filled edible capsule.
